(19) United States
(12) Patent Application Publication
(10) Pub. No.: US 2013/0244895 A1
(43) Pub. Date: Sep. 19, 2013

(54) Microwell Arrays for Direct Quantification of Analytes on a Flat Sample

(71) Applicants: Janos Vörös, Zurich (CH); Marcy Zenobi-Wong, Zurich (CH); Prayanka Rajendra, Zurich (CH)

(72) Inventors: Janos Vörös, Zurich (CH); Marcy Zenobi-Wong, Zurich (CH); Prayanka Rajendra, Zurich (CH)

(73) Assignee: ETH ZURICH, ETH TRANSFER, Zurich (CH)

(21) Appl. No.: 13/817,980

(22) PCT Filed: Aug. 18, 2011
(86) PCT No.: PCT/EP2011/004150

§ 371 Date: May 1, 2013

(30) Foreign Application Priority Data
Aug. 20, 2010 (EP) 10008715.4

(51) Int. Cl. G01N 33/543

(52) U.S. Cl. CPC G01N 33/54373
USPC 506/9; 506/39

(57) ABSTRACT

The present invention relates to a bioanalytical device consisting of a microwell array with microwell (2) that are filled with assay components (12, 15, 36), wherein detection probes (36) used in the assay (10) are metal nanoparticles (11, 12) or fluorescent compounds, and wherein the microwell array is connected and/or connectable to a sample that is on a flat substrate (6) to quantify the amount of a ligand (35) in the sample by using a detection mechanism. The detection mechanism is based on change in the optical properties of some of the assay components (12, 15, 36) upon contact with the ligand (35). The present invention also relates further to a method for detecting and quantifying molecules using said bioanalytical device.

### TECHNICAL FIELD

The present invention relates to a bioanalytical device for detection and quantification of analytes on a flat sample, and to a corresponding method.

### PRIOR ART

Cavity arrays have been used to determine the bulk concentration of nanometer sized objects such as lipid vesicles by dispensing them into individual wells. Another technology, the so called digital microarrays from Oxford Gene Technology (http://www.ogt.co.uk/) dispenses cells to be investigated into individual wells, but then a five step assay is run on them to achieve the results.

In addition, several microfluidic approaches exist for the analysis of the genetic content of cells in microfluidic devices, but these generally involve PCR to bring the amount of DNA to a detectable level.

### SUMMARY OF THE INVENTION

It is an object of the present invention to provide a bioanalytical device allowing for a more accurate and efficient detection and/or quantification of analytes, in particular of nucleic acids. This object is achieved by a bioanalytical device having the features of claim 1. A further object of the present invention is to provide a method for detecting said molecules using said bioanalytical device. The latter object is achieved by a method having the features of claim 10.

The above mentioned objects are achieved and the problems solved by the present invention that provides a bioanalytical device consisting of or comprising a microwell array filled with assay components. The detection probes used in the assay are preferably metal nanoparticles and/or fluorescent compounds. Here, the assays are preferably one pot assays. The microwell array is connected and/or connectable to a sample that is on a flat substrate to quantify the amount of a ligand or said molecule in the sample using a detection mechanism, wherein the detection mechanism is based on a change in the optical properties of some of the assay components upon contact with the ligand. A corresponding method is proposed for detection and/or quantification of said ligand.

The expression “bioanalytical device” includes situations where such a bioanalytical device is a subunit of a complete bioanalytical device. A complete analytical device may include or comprise further elements. These further elements may be control elements, tubes, supports, chemicals, tools or other equipment that a person skilled in the art of bioanalysis with microwells regards as important.

A microwell array comprises at least one or more individual microwells, it may even comprise up to thousands of microwells. The microwell array is preferably a PDMS microwell array, preferably provided on a glass substrate. Said glass substrate may also be provided with a plurality (e.g. 5 to 100 or more) of individual microwell arrays. Preferably, the dimensions (i.e. length, width, and depth; or diameter and depth or height) of the microwells in the array are 100 nanometer to 1 millimeter, more preferably between 1 micrometer and 100 micrometer, more preferably between 10 micrometer and 50 micrometer. Furthermore, said microwells preferably have a cylindrical shape. It is advantageous, if said microwells are not in fluid-communication with one another. This avoids contamination and allows for multiplexing. Such a microwell is filled with or comprises a detection assay. In this context, the term “filled” or “filled with” is to be understood as completely or partially filled. Completely filled is a microwell, if essentially all its volume is taken by the filling substance. Partially filled means that the microwell is only filled up to e.g. 1/10 to 9/10 of its volume or height (or depth). This detection assay includes detection assay components. Under the microwell being connected and/or connectable to a sample that is on a flat substrate includes situations in which e.g. the microwell has an opening such that a sample on a flat substrate, preferably in a dried state, can be brought in contact, preferably in fluid-communication, with said detection assay through said opening. This opening is preferably an upwardly open part of the microwell. Through said contact, the sample enters the detection assay. To improve this process, the detection assay may be heated, e.g. up to 90 degree Celsius. Said detection assay may be based on the sequence specificity with respect to the target ligand of interest and may include (functionalized) detection probes for detecting said ligand and quantifying its amount in the sample. It is proposed to use a detection mechanism based on a change in the optical properties of some of the assay components upon contact of the (functionalized) detection probes with said ligand.

The detection probes used in the detection assay include nanoparticles or fluorescent compounds. A nanoparticle is a particle with essentially spherical shape, wherein this sphere has a radius in the nanometer range (e.g. 1 nanometer to 1 micrometer). Situations are included in which the nanoparticles may deviate from a spherical shape, being rather like an ellipsoid of revolution or having recesses, i.e. shapes as known from existing metal colloids. It is also contemplated that plate-like nanoparticles may be used, if the corresponding optical change upon coupling is measurable. Preferably, the nanoparticles are based on or made of metal, more preferably based on or made of a noble metal, e.g. based on or made of gold and/or silver. The nanoparticles have preferably a diameter of up to and inclusive of 200 nanometer, more preferably of around 50 nanometer. The fluorescent compounds include preferably a dye and/or a protein, e.g. quantum dots, fluorescent dye molecules, fluorescent beads, fluorescent microspheres.

A detection probe may be functionalized or modified by tagging or decorating it with a nucleic acid or an oligonucleotide. Preferably, the detection probes are tagged with thiolated DNA or with thiolated oligonucleotides. They are thereby adapted to couple to a predefined part of said target ligand, preferably by hybridization. The tag is chosen such that the functionalized detection probe, e.g. the nanoparticle or the fluorescent compound, couples to the specifically chosen predefined part of the target ligand. The target ligand itself is a biomolecule, in particular a protein, RNA, DNA, an oligonucleotide, a carbohydrate, or a lipid, a small molecule, or a cell fragment, preferably of a single cell. Preferably, the sample is a cell culture or a spotted microarray. It can be an array of different cell cultures or cells in an array. Parts of the individual wells may also be filled with different assay components in order to detect and/or quantify different ligands in neighboring microwells or the same ligands with different detection probes. In some embodiments of the present invention, the assay components include compounds such as Tris-Cl, NaCl, MgCl₂, RNase inhibitor, dithiothreitol, phosphate buffered saline which are suitable for the lysis of the sample, which frees the target analytes.

Preferably, detection assay components or assay components in the microwell include a phosphate buffer, preferably a sodium phosphate buffer. Said phosphate buffer is included preferably in an amount in the range of 9 mg (ml)⁻¹ [milligram per milliliter] to 11 mg (ml)⁻¹, more preferably of 9.8 mg (ml)⁻¹. The assay components may further include sodium chloride, preferably in an amount in the range of 3 mg (ml)⁻¹ to 9 mg (ml)⁻¹, more preferably of 6 mg (ml)⁻¹ or in the range of 200 mM to 1000 mM, preferably in the range of 300 mM to 800 mM. The assay components may further include detection probes, preferably (metal) nanoparticles in an amount in the range of 4.5×10⁸ to 7×10¹¹ colloids per ml, more preferably of 7×10⁹ to 4.5×10¹⁰ colloids per ml or fluorescent molecules or compounds in the range of 3×10¹⁰ to 6×10¹⁷ molecules per ml, more preferably of 4.8×10¹⁵ molecules per ml, preferably in water or a water-based liquid. These above mentioned detection probes are tagged to the oligonucleotides covalently prior to the assay. A density of colloids per milliliter may be adjusted by addition of HEPES buffer to the colloid solution. In order to prevent drying out of the microwell, the assay components may further include glycerol in the range of 10% to 60% by volume, more preferably around 30% by volume.

In some embodiments of the present invention, a first fraction, preferably about 40% up to 60% of the nanoparticles, more preferably about 50%, i.e. half of the nanoparticles or the fluorescent compounds or the detection probes, respectively, in the detection assay are functionalized or modified, preferably with predefined thiolated oligonucleotides or with other systems as mentioned above or below, such that said first fraction of functionalized nanoparticles or the fluorescent compounds, respectively, couple to a first predefined part of the target ligand by hybridization. A second fraction of the nanoparticles or the fluorescent compounds or the detection probes, respectively, in the detection assay is functionalized, preferably with predefined thiolated oligonucleotides or with other systems as mentioned above or below. Said second fraction may be 60% down to 40% of the nanoparticles, preferably around 50% of the detection probes. Said second fraction of the functionalized nanoparticles or fluorescent compounds, respectively, couple to a second predefined part of the target ligand, preferably by hybridization. Said second part of the target ligand is located close to said first part of the target ligand, so that the distance between the nanoparticles and/or the fluorescent compounds, respectively, which are coupled to the same target ligand is small, such that the coupled nanoparticles or fluorescent compounds, respectively, couple optically. Said distance is preferably smaller than or equal to 5 nanometer.

Situations are included in which said first fraction and said second fraction preferably supplement to 1, i.e. each detection probe (e.g. nanoparticle and/or fluorescent compound) is either in the first or the second fraction. Preferably, the number of detection probes is the same in each fraction, and preferably as many probes as possible take part in the coupling reaction. Alternatively, the first and second fractions include only 80% to 99% or less of all detection probes.

The term “optically coupled” means that an optical property, such as a color of at least part of the assay or such as entire optical spectra, changes measurably. The color change may even be visible by naked eye. The optical spectra are preferably recorded by means of a spectrometer or similar means.

According to another embodiment of the present invention, the bottom of the microwell, i.e. the substrate on which the microwell structure is placed, is decorated with nanodisks. These nanodisks are preferably based on or made of a metal, in particular based on or made of a noble metal such as gold and/or silver. Said nanodisks may have a diameter between 10 nanometer and 10 micrometer, more preferably between 50 nanometer and 300 nanometer, and even more preferably of around or about 110 nanometer. The thickness of said nanodisks is preferably in a range of 10 nanometer to 100 nanometer, preferably of about 30 nanometer. Situations are included, in which the thickness may be adjusted to the diameter of the nanodisk, so that said thickness is e.g. 1/10 to ⅔ of said diameter. The size of the nanodisks may be adjusted to the size of the detection probes used. Said nanodisks preferably have a distance from one another of preferably around 300 nanometer. This distance may be larger or smaller, i.e. 500 to 1000 nanometer or 10 to 100 nanometer, respectively, depending on the sample and the size of the nanodisks. These disks are generally flat platelets of an arbitrary but preferably substantially circular or rectangular shape. The nanodisks are functionalized, preferably with thiolated oligonucleotides or with another system as outlined above or below, such that the functionalized nanodisks are adapted to couple to a first predefined part of the target ligand, preferably by hybridization. At least part of the nanoparticles in the detection assay are functionalized, preferably with predefined thiolated oligonucleotides or with another systems as outlined above or below, so that said functionalized nanoparticles couple to a second predefined part of the target ligand, preferably by hybridization. Said second part of the target ligand is located close to said first part of the target ligand. Therefore the distance between said nanodisk and said nanoparticle coupled to the same target ligand or the distance between two or more coupled nanoparticles is small, such that the coupled nanoparticles or fluorescent compounds, respectively, couple optically. Said distance is preferably smaller than or equal to 5 nanometer.

The present invention further includes a method for detecting and quantifying molecules by use of said bioanalytical device. In this method, an optical property of at least a part of the detection assay in the microwell array, said array including the sample and the detection probes, is determined. The sample includes the target ligand of interest and the detection probes are adapted to couple to said target ligand, wherein an optical response of the detection assay is changed, as outlined above or below. The optical property is preferably a color, which is visible by eye, or optical spectra. Here, spectrometer means are applied for recording of said optical spectra. Said color or said optical spectra, respectively, are determined, preferably at room temperature, after the ligand to be detected and/or quantified in the sample has coupled to the respective detection probes, preferably after mixing or heating the detection assay and the sample at or to preferably 90 degree Celsius. A quantity of ligand in the sample is determined by comparison of said color or said optical spectra, respectively, with a reference. Said reference is preferably a color of or optical spectra recorded for a reference assay with a known ligand quantity, respectively. Situations are included in which also computer simulations or documented previous measurements are a possible reference.

In an embodiment of the method according to invention, the flat substrate on which the sample is provided is preferably at least part of a coverslip being used for covering the microwell that includes the detection assay. The sample is preferably a cell culture, a spotted microarray, a single cell, or a small number of cells and applied to the coverslip, preferably in a dried state. Upon covering the microwell with the coverslip, the sample gets into contact, preferably but not necessarily in fluid-communication with the detection assay and distributes therein. The distribution of the detection probes is preferably substantially homogenous over the whole detection assay. The sample couples in said detection assay with the detection probes, wherein the target ligand in the sample is preferably a biomolecule, in particular a protein, RNA, DNA, an oligonucleotide, a carbohydrate, or a lipid, a small molecule, or a cell fragment.

Measuring the optical spectra includes preferably measuring a fluorescent response or a surface plasmon resonance of the detection probes in the detection assay. The localized surface plasmon resonance is measured in a light wavelength range from preferably 400 nanometer to 800 nanometer. Then, preferably an extremum in the intensity, more preferably a maximum, of said surface plasmon resonance is determined, e.g. a scattering intensity or extinction maximum, Said comparison with similar optical spectra from the reference assay includes in particular comparing said extrema, wherein a wavelength shift of said extrema can be used as a measure for the quantity of the target ligand in the sample. In some embodiments, measuring the optical spectra may include measurements of a transmission or a scattering intensity of preferably visible light (range e.g. 400 to 800 nm) transmitted through or scattered by the assay. Preferably, the shape of the microwell of the used microwell array is optimized to have a shape of a long cylinder for transmission intensity measurements or to have a disk- or plate-like shape for scattering intensity measurements, respectively. A microwell array may also comprise differently shaped microwells.

Plasmonic nanoparticles are a powerful tool to study biomolecular interactions with high sensitivity and specificity. Among the several biomolecules (nucleic acids, protein, lipids, carbohydrates etc.) that can be detected using the reported assay, oligonucleotides are of special interest as they can unveil the understanding at the molecular level of the cells. Direct oligonucleotide quantification assays can be more attractive than other comparative scale detection systems since they offer the potential to monitor single cells without the use of reverse transcription and amplification steps as required for Reverse Transcription-Polymerase Chain Reaction (RT-PCR). Although PCR, DNA microarrays etc. have already become standard methods for the detection of nucleic acids, these methods still lack accuracy and are rather cumbersome which hampers a commercial breakthrough for oligonucleotide detection technology. The present invention shall improve the applicability of oligonucleotide detection technology.

The use of noble metal nanoparticles such as e.g. gold (Au) and silver (Ag) colloids shall now be explained in more detail. These nanoparticles exhibit distinct localized surface plasmon resonance (LSPR) frequency which is dependent on the surrounding environment and which makes them useful as particle sensors. The LSPR frequency of these metal nanoparticles is unique and depends not only on the metal, but also on the size and shape of the nanoparticle, the dielectric properties of the local medium, and inter-nanoparticle coupling interactions, thus imparting the possibility to tune the photophysical properties of the nanoparticle. Its unparalleled feature of high sensitivity to the change in refractive index of the surrounding medium has been exploited for sensing and diagnostics applications.

A shift in the SPR wavelength of a metal nanoparticle induced by the absorption of an analyte is primarily caused by a change in the local dielectric environment, although this shift is not specific to the chemical or biological species being adsorbed. If this property can be used for biosensing, the specificity must be achieved by the presence of surface ligands which are specific to the analyte molecule of interest and which discriminate nonspecific surface adsorption. This specificity can be demonstrated using the well-established streptavidin-biotin system. These biosensing platforms can be later extended to optically detect glucose levels, antibody-antigen interactions and oligonucleotide hybridization studies. The detection limit in such assays can be pushed down by reducing the number of nanoparticles being probed, even to single-nanoparticle level. For single particle spectroscopy studies, scattering techniques offer a greater advantage if compared to absorption spectroscopy that suffers from the low signal-to-noise ratio. By combining dark-field illumination with spectroscopy, the scattering spectrum of single nanoparticle can be collected with a very high signal to noise ratio. For such single particle probing, metal nanoparticles with a high scattering quantum yield are chosen based on their well-characterized optical properties.

Plasmon coupling interaction between metal nanoparticles is of important consideration too as this forms the basis for nanoparticle assembly with biomolecules (DNA, RNA etc.) as the linkers. The extent of resonant peak shift induced due to the nanoparticle coupling is also dependent on the distance between the interacting nanoparticles and its orientation with respect to each other.

In some embodiments, a fluorescent signal of the detection probes in the detection assay is measured, wherein said fluorescent signal changes upon said coupling between the target ligand and the detection probes, and wherein said change can be used as a measure for the quantity of the target ligand in the sample.

The use of fluorescent molecules shall now be described in more detail. It is also a distance based detection method that can be applied to detect oligonucleotides. Fluorescence resonance energy transfer (FRET) is widely used in biomedical research as a reporter method. Oligonucleotides labelled with one or several fluorophores can form FRET systems. FRET can occur when the duplex is formed between two labelled oligonucleotides, bringing the donor and acceptor dyes in close proximity. Alternatively, the hairpin configuration of the oligonucleotide dual-labelled with both donor and acceptor fluorophores can result in FRET. The detection of FRET and its disruption can be both used in the assays. The feasibility of FRET implies that the two fluorophore molecules are physically within a few nanometers. FRET disruption indicates that the relative positions of the molecules have changed and the new distance between them is prevents the occurrence of energy transfer. Several FRET-based molecular probes such as molecular beacons [19] and TaqMan probes, the fluorescence signals of which change as a result of hybridization or enzymatic reactions, exist to enable detection of DNA. When FRET occurs, the donor fluorescence intensity decreases and the acceptor fluorescence intensity increases which can be detected spectrally. Such fluorescent assays also have a potential to be carried out in the present invention and the changes in the spectral properties can be studied with the spectrometer coupled with the light source that can excite the donor molecule alone.

Preferably, the method includes measuring each microwell individually, preferably at the same time. This is possible, since the detection mechanism is imaging based, hence allowing for parallel detection of multiple samples. This multiplexing option saves a lot of time and effort.

Combing the microwell array system filled with all components for the biochemical detection assay is advantageous compared to earlier methods as most samples are already on a flat format (e.g. cell cultures, tissue slices, and spotted microarrays), hence there is no need for fluidic systems to harvest the sample before analysis. It is further advantageous that the dimensions of the microwells define the total volume of the assay, hence the sample dilution and the reagent consumption is minimized. This allows for measurements of the target ligand content of a single cell without the need for amplification, in particular, there is no need for PCR. Further, as all the detection assay components are preloaded to the microwells, fluidic components are unnecessary and hence advantageously eliminated. The individual wells are well separated (e.g. about 50 micrometer or more wall-to-wall spacing, which avoids also lateral connection and direct fluid-communication between the individual microwells). The microwells can therefore be read-out individually, wherein different detection or quantification is done in neighboring microwells, which provides excellent multiplexing possibilities.

The present invention thus provides a device consisting of a microwell array filled with suitable assay components and connected to a sample that is on a flat substrate to quantify the amount of a suitable ligand in the sample using a suitable detection mechanism. Said device can be used to analyze the content of biological samples, in particular those which are on a flat substrate, such as cell cultures or microarrays. It is especially suited for the analysis of e.g. the protein, RNA or mRNA, DNA, lipid, or sugar content of single (or a small number of) cells, using various reporter probes such as plasmonic nanoparticles, fluorescent molecules etc. as biosensors. Preferably, the (target) ligand is a biomolecule (for example a protein, an oligonucleotide, a carbohydrate, or lipid), a small molecule, or a cell fragment. The microwells of the device according to invention have dimensions (i.e. length, width, and depth) of preferably 100 nanometer to 1 millimeter, more preferably between 1 micrometer and 100 micrometer. Preferably, the detection assay components include compounds which are suitable for the lysis of the sample. The detection mechanism is based on change in the optical properties (e.g. fluorescence, scattering, absorption, or color) of some of the assay components upon contact with the ligand. Preferably, the sample is a cell culture or a spotted microarray. Preferably, part of the individual microwells is filled (completely or partially) with different assay components. Preferably, the detection mechanism is imaging based making parallel detection of multiple samples (e.g. cells) possible. Preferably, the detection probe used in the assay is either metal nanoparticles (for example gold, silver) or fluorescent compounds (for example a dye, protein). Preferably, the detection assay in the microwells is based on the sequence specificity with respect to the ligand.

A sensitive and sequence-based direct detection of oligonucleotides is advantageous for quantifying the amount of DNA/RNA in a single cell. In general, direct oligonucleotide quantification assays are attractive detection systems since they offer the potential to monitor single cells without the use of amplification steps such as reverse transcription and amplification steps as required for Reverse Transcription-Polymerase Chain Reaction (RT-PCR). The present invention reduces the risk of contamination and eliminates time-consuming intermediate steps. Based on these advantages a spectral based (e.g. scattering or transmission) oligonucleotide detection system with thiol-functionalized oligonucleotide-modified 50 nm gold probes is proposed as one embodiment of the present invention. In this system, oligonucleotide-modified gold nanoparticles are dispersed into the microwells (e.g. cylindrically-shape with a 100 or 300 micrometer diameter and 20 micrometer height or depth). According to one embodiment of the present invention, the target analyte is suspended in sodium chloride (NaCl, e.g. 300 mM to 800 mM), in about 10 mM phosphate buffer and about 30% glycerol by volume, the latter to prevent the microwells from drying out. The target analyte is dispersed into the microwells or spotted onto a glass surface, preferably a coverslip. Said coverslip is then used to cover a microwell containing a corresponding detection assay, and placed on the microarray structures, which contains the gold nanoparticles. Thus the target analyte or sample comes into contact with or is connected to said assay. In the presence of the complementary target oligonucleotide the assay results in gold nanoparticles forming pairs and/or aggregated polymeric networks via side-by-side hybridization events between two oligonucleotide probes. The hybridization events induce aggregation which leads to concomitant change in the optical spectra (e.g. scattering or extinction spectra) of the nanoparticles which can be utilized for the detection and quantification of e.g. nucleic acids, in particular DNA or other target ligands. The distinct light scattering properties of the gold nanoparticles can be utilized for the detection and quantification of DNA. The binding of the analyte (e.g. target DNA or RNA molecule) to the functionalized, preferably oligonucleotide-modified gold nanoparticles during the assay brings the plasmonic nanoparticles in close vicinity to each other (<5 nanometer). Due to this proximity effect they become optically coupled, creating a more enhanced field and giving a strong resonance depending on the coupling strength or interparticle distance. As a result there is a second scattering or extinction peak towards the red region, i.e. at longer light wavelengths.

A microwell array system is proposed to carry out this assay. This makes it possible to detect directly, in particular without any previous PCR steps, DNA in naturally occurring quantities. The present invention is particularly interesting for quantifying the RNA or an mRNA copy number from single cells without the need for sample/signal amplification. This assay system allows for the direct detection of DNA or RNA in naturally occurring quantities and may be used to quantify gene expression from single cells, living, fixed or lysed, without the need for sample and/or signal amplification. The concentration of oligonucleotides in a cell is not too low in comparison with the volume of a single cell. Even a few copies of mRNA in a single cell will be a concentration possible to quantify. A main focus of the present invention is to prevent the dilution of the sample to be studied. For the measurements, a custom-made dark field spectro-microscope can be used which combines a spectrometer coupled to an inverted optical microscope with halogen lamp illumination and a CCD camera.

An aim of the present invention is to use a microwell array system as a single pot for quantification of an analyte of interest preferably in one step, based on a detection method (fluorescence, scattering absorption, color etc.) provided that it generates signals that are proportional to the number of one or more specific analytes in the sample. Gold nanoparticles will be the primary label considered, although it is foreseen to alternatively of additionally use hybrid/alloy structures of other (noble) metal nanoparticles, fluorescent dyes, quantum dots, or other detection probes for multiplexing detection. Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1a,b shows wavelength shift in the presence (red) and absence of target sequence (black); specifically FIG. 1a schematically shows a microwell before coupling of the probes and the target ligand; FIG. 1b shows the situation after the coupling;

FIG. 2 shows a peak position shift due to various binding events from a sandwich assay in a flow cell;

FIG. 3 shows a spectral shift in the scattering intensity;

FIG. 4a shows schematics of an assay performance with gold nanoparticles; upon target binding the gold nanoparticles get close to each other generating a new coupled plasmonic mode in their optical spectra;

FIG. 4b shows absorption spectra of single particles in air and in buffer;

FIG. 4c shows absorption spectra of coupled particles in air and in buffer;

FIG. 5 shows a schematic representation of a PDMS well array on a flat glass substrate;

FIG. 6 shows schematics of a model microwell for a transmission (left) and scattering (right) mode of spectral studies;

FIG. 7 shows a microscopy image of four selected PDMS microwells with 150 μm diameter;

FIG. 8 shows schematics of a gold nanoparticle assay in microwells for direct DNA quantification (top) and the model of their corresponding scattering spectra (bottom);

FIG. 9 shows scattering spectra of coupled particles heated to different temperatures recorded at room temperature;

FIG. 10 shows the relation between the effect of the heating step and the number of coupled particles;

FIG. 11 shows scattering spectra of gold nanoparticles with varying number of target molecules heated to 90° C. and measured at room temperature;

FIG. 12 shows the relationship between the number of target DNA added and the calculated number of coupled particles;

FIG. 13 shows SEM images of gold nanodisks of 110 nm diameter immobilized with gold colloids (50 nm diameter) due to DNA hybridization (Right: SEM image with a 60° tilt);

FIG. 14 shows scattering spectra of gold nanodisks with varying number of target molecules in the microwells; and

FIG. 15 shows schematics of the assay performed in microwells; the microwell also contains all assay components that might be advantageous e.g. to lyse the cells and free the target analytes.

### DESCRIPTION OF PREFERRED EMBODIMENTS

As one embodiment, a sensitive and selective sequence-based sensing method for DNA is proposed, using plasmonic gold particles 11, 12 and/or fluorescent compounds in microwell array 1. The distinct light scattering properties of the gold nanoparticles 11, 12 and/or the distinct fluorescent signal can be utilized for the detection of DNA or nucleic acids (cf. R. A. Reynolds, C. A. Mirkin, and R. L. Letsinger, (2000) J. Am. Chem. Soc. 122, 3795-3796).

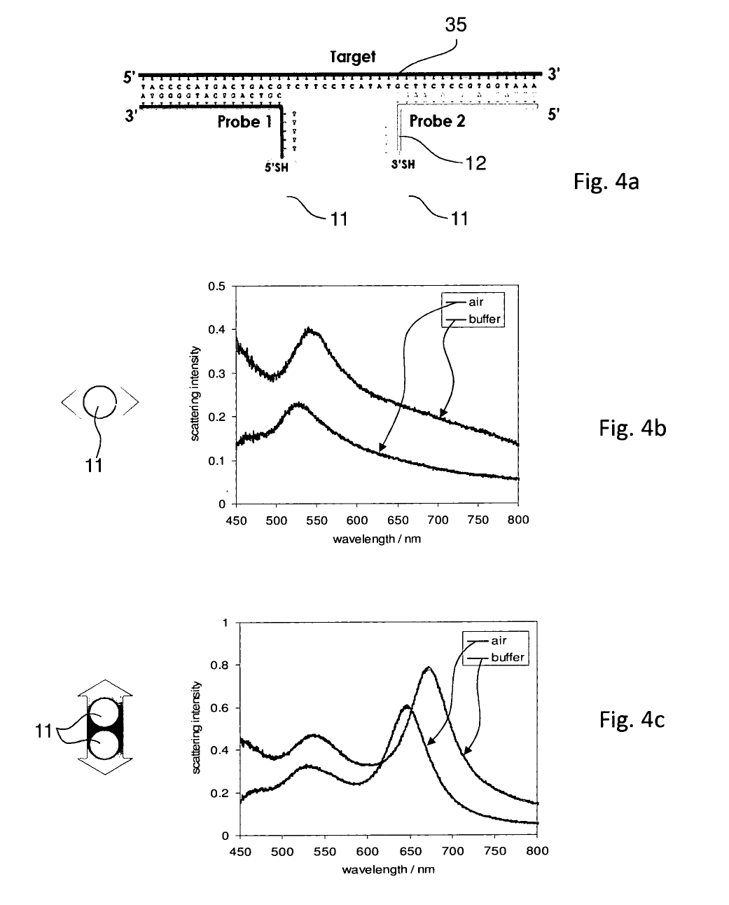

The binding of the analyte 35 to the gold nanoparticles 12 (cf. FIG. 4a) in this detection assay 10 leads to a local refractive index change. In other words, the binding of the analyte 35 to at least two differently functionalized gold nanoparticles 12 (cf. FIG. 4a) leads to a drastic change in the plasmonic spectra of the functionalized particles 12 (cf. FIGS. 4b and 4c). This increase in refractive index accounts for a wavelength red-shift of the nanoparticles extinction maximum. This assay made in a microwell array 1 (for production details of the microwell array specific reference is made to: A. Binkert, P. Studer, and J. Voros (2009), Small, 5, 1070-1077, the disclosure of which is incorporated) (cf. FIG. 7) makes it possible to detect DNA in naturally occurring quantities in very small volumes. The 50 nm gold colloids 11 (4.5×10¹⁰ to 7×10¹¹ colloids ml⁻¹) (GC50, British Biocell, UK) were tagged with thiolated-DNA (Probe 1 and Probe 2, see Table 1) (Eurogentec, Belgium) by the process described in T. Sannomiya, C. Hafner and J. Voros, (2008) Nano letters, 8, 3450-3455. An example of a target ligand 35, labeled as Target, is given in the table 1 below. Table 1 further includes the two modification tags Probe 1 and Probe 2. Probe 1, tagged to a fraction, preferably half of the nanoparticles in the detection assay 10, and Probe 2, tagged to at least another fraction, preferably to all other nanoparticles in the detection assay 10.

TABLE 1
DNA Sequences.

| Name | Modification | Sequence 5′-3′ |
|---|---|---|
| Probe1 | Thiol-C6 (5′) | TTT-TTT-TTT-TGA-GAG-ACC- |
| (Seq. D1) | | GGC-GCA-C |
| Probe2 | Thiol-C6 (3′) | TTG-TGC-CTG-TCC-TGG-TTT- |
| (Seq. ID2) | | TTT-TTT-T |
| Target | | GTG-CGC-CGG-TCT-CTC-CCA- |
| (Seq. ID3) | | GGA-CAG-GCA-CAA |

The colloid solution was first mixed with an equal amount of water based DNA solution for 24 h. Then, 9.8 mg (ml)⁻¹ phosphate buffer 15 and 6 mg (ml)⁻¹ NaCl were added. After 48 h the final concentration of detection probes 36 in the detection assay 10 in form of the DNA tagged gold colloid solution was adjusted via centrifugation of the gold colloid solution using 14000 g for 10 minutes, removal of the supernatant and addition of HEPES buffer to adjust the concentration of approximately 1/100 of the original colloid concentration.

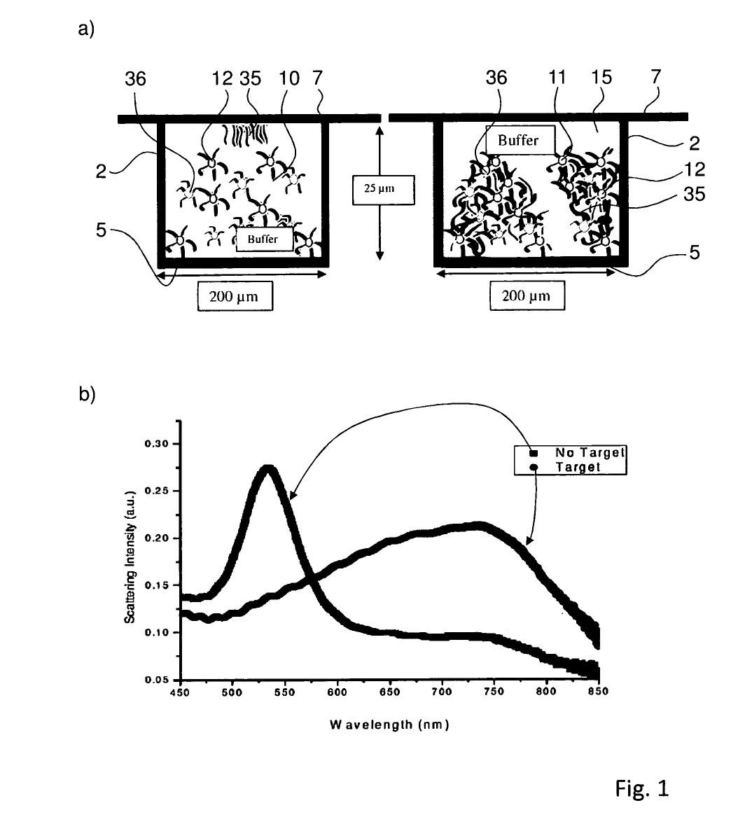

A glass substrate 5 was used with a microwell array 1 as described below. The microwells 2 have a cylindrical shape with a diameter of 200 micrometer and a depth of 25 micrometer (cf. FIG. 1, top). Said glass substrate 5 with microwell structure was first plasma-cleaned to render the hydrophilic property. Then, the microwells 2 were filled with the detection assay 10, including the mixture of Probe 1 and 2. The microwell array 1 is then covered with a coverslip 7 on which a sample 30 with the Target probe or ligand 35 is dried (cf. FIG. 1, top left). After the detection probes 36 are coupled to the target ligand 35 (cf. FIG. 1 top right), optical measurements are carried out. The complete spectral recording was done carefully without letting the wells 2 to dry. The differences or change in spectra in the presence and absence of target 35 were recorded.

The spectral measurements were conducted by a custom built microscope (Axiovert 200, Zeiss, Germany) with a spectrometer (SpectraPro 2150, PIXIS 400, Princeton Instruments, US). The online data analysis and the control of the spectrometer were carried out by a custom made program.

In FIG. 1b, the change in λₘₐₓ as the result of target binding to Probe 1 and Probe 2 is shown. λₘₐₓ is the wavelength at which a maximum in the scattering intensity occurs. A high ionic strength buffer (e.g. 800 mM NaCl) is used for successful hybridization to the probes. Under these conditions aggregation is maximum as shown from the shape of the curve in FIG. 1b. The Probes 1 and 2 aggregate due to the target presence, resulting in a second peak at λ>600 nm.

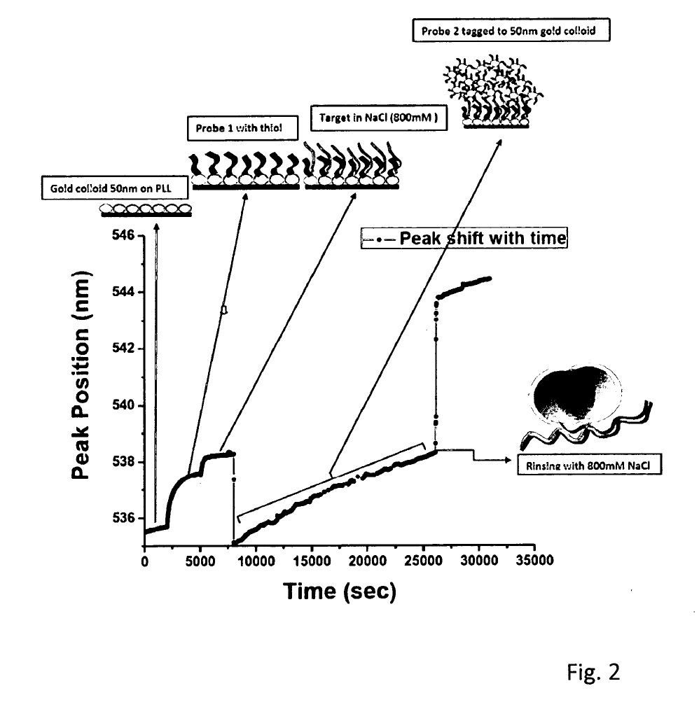

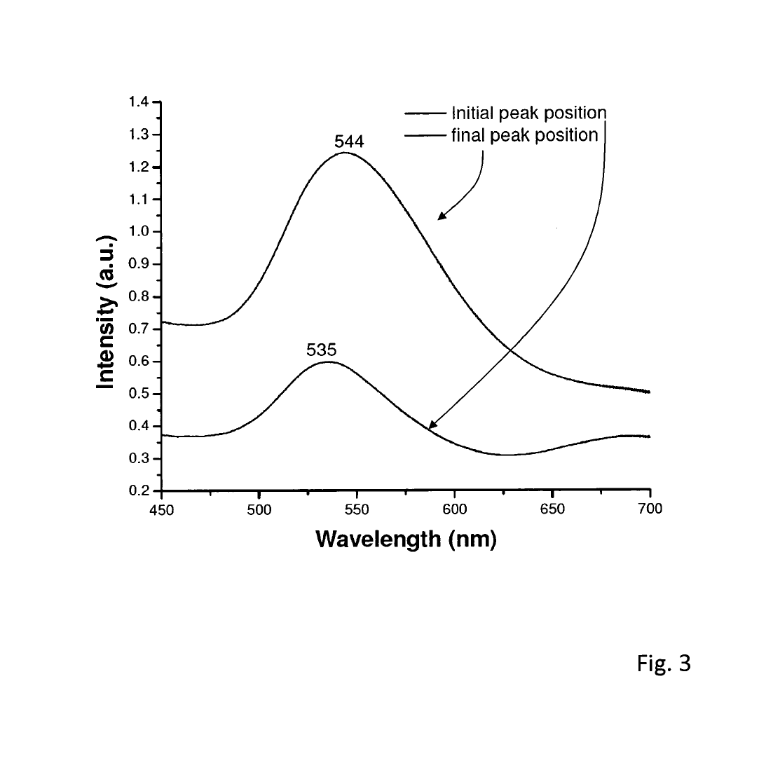

The results shown in FIG. 2 are from the same detection assay 10 performed in a flowcell. FIG. 2 shows peak position shift due to various binding events starting from the initial peak position of the spectrum firstly when the gold colloid is coated over the layer of PLL followed by the covalent binding of the thiol DNA then the target hybridization to the thiol DNA proceeded by the hybridization of the thiol functionalized gold nanoparticles to target and finally the rinsing of the unbound gold colloids. FIG. 3 displays the whole spectrum, showing the shift in the scattering intensity during the initial and the final steps of the assay.

The DNA hybridization as outlined just above relies on the possibilities of sandwiching a target ligand 35 between two functionalized gold nanoparticles 12 to form a complex, which results in a considerable wavelength shift. It may be used to quantify the RNA from single cells without amplification.

In another embodiment, citrate-stabilized 50 nm gold nanoparticles (11) (with a density of 4.5×10¹⁰ colloids (ml)⁻¹) (GC50, British Biocell, UK) were functionalized with thiolated oligonucleotides by incubating the gold dispersion with disulfide-protected oligonucleotides (thiol-ssDNA 100 nmol(ml)⁻¹ of gold colloid) in aqueous solution, overnight. The thiolated oligonucleotides Probe 1 and Probe 2 can have the thiol functional group either on the 3′ or on the 5′ based on the desired orientation of the gold colloids to be studied (head to head, head to tail or tail to tail). This also applies for the functionalization or modification of other nanoparticles and/or other detection probes, if applicable. The probe 1 and probe 2 sequences can be about 10 to 40 bp long including the polytail at the thiol terminal which can range from 5 to 10 bp long. The polytail can be composed of any one of the four nucleotide bases A, T, G or C. The dispersion was brought to a final salt concentration of NaCl (300 mM) and sodium phosphate buffer 15 (10 mM, pH 7.4), and the unbound oligonucleotides were removed by repeated centrifugation and redispersion of the pellet. In addition, the concentration of DNA in the supernatant separated after centrifugation was too low to measure, which implies that the loss of material was minor. The DNA-complexed gold nanoparticles 12 (ssDNA-thiol-NP) were stored in NaCl (300 mM) and sodium phosphate buffer (10 mM, pH 7) for further use. The assay is performed when an equal number of Probe 1 and 2 tagged gold nanoparticles (see Tab. 2) are mixed with target oligonucleotide and heated to 90° C. Then spectral studies were performed at room temperature.

TABLE 2
DNA Sequences of p53.

| Name | Modification | Sequence 5′-3′ |
|---|---|---|
| Probe1 | Thiol-C6 (5′) | TTT TTT TTT TGA GAG ACC |
| (Seq. ID4) | | GGC GCA C |
| Probe2 | Thiol-C6 (3′) | TTT TTT TTT TTT GTG CCT |
| (Seq. ID5) | | GTC CTG G |
| Target | None | GTG CGC CGG TCT CTC CCA |
| (Seq. ID6) | | GGA CAG GCA CAA |

The sequence p53 mentioned above is shown to be a tumour suppressor gene and the sequence was adopted from the previous work of Tao. H , Wei. L, Liang A., et al., Highly Sensitive Resonance Scattering Detection of DNA Hybridization Using Aptamer-Modified Gold Nanopaticle as Catalyst, Plasmonics (2010) 5: p189-198.

A general overview of the oligonucleotide design:

| | | | Total length |
|---|---|---|---|
| Name | Modification | Polytail length | of sequence |
| Probe1 | Thiol-C6 (5′)/Thiol C6 (3′) | 5-15 bp long | 10-40 bp |
| Probe2 | Thiol-C6 (5′)/Thiol C6 (3′) | 5-15 bp long | 10-40 bp |
| Target | None | None | Regions |
| | | | complementary |
| | | | to the Probe 1 |
| | | | and Probe 2 |
| | | | with a |
| | | | difference |
| | | | in melting |
| | | | temperature |
| | | | (Tm) not greater |
| | | | than 1-2° C. |

A Microwell System with Purified Oligonucleotide (PDMS Microwell Fabrication)

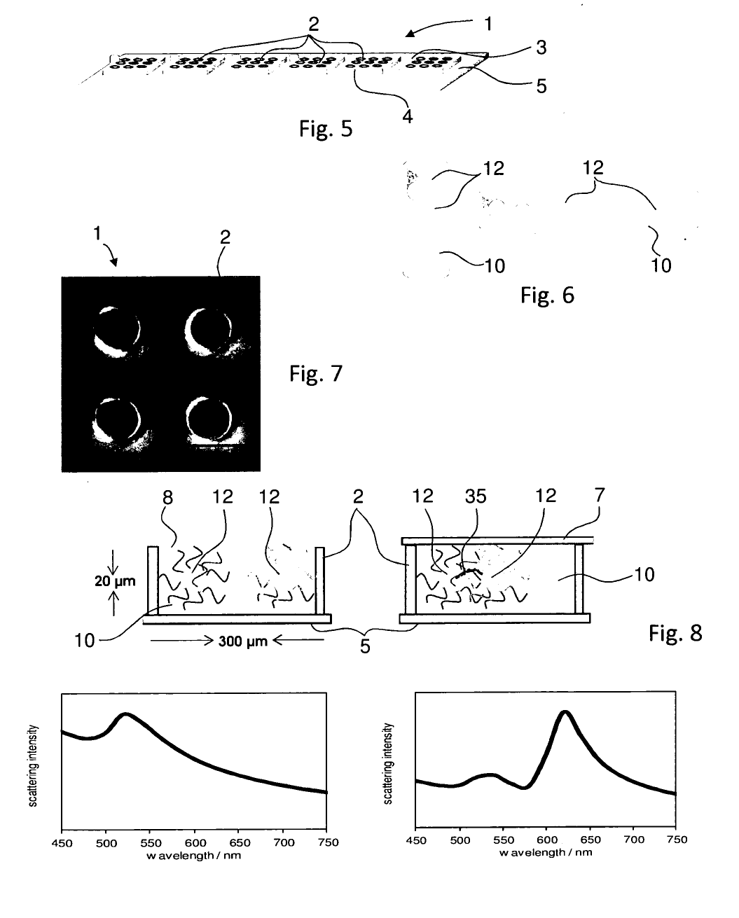

As for the fabrication of the microwell structure specific reference is made to: Binkert, A., P. Studer, and J. Voros, A Microwell Array Platform for Picoliter Membrane Protein Assays. Small, 2009. 5(9): p. 1070-1077, the disclosure of which is incorporated. For the fabrication photolithography of SU-8 patterns on a glass slide (GM 1070, Gersteltec) was used. This master is further used to create a negative template in poly (dimethylsiloxane) (PDMS) (Sylgard 184, Dow Corning), the structure being cast in a PDMS slab with 6 arrays (cf. FIG. 5). The spacing, i.e. the width of walls 4, and diameter of the microwells 2 (columns in the master) were varied according to the requirements. Also, PDMS ports 3 are shown, which are the points where the fresh PDMS was poured through the negative PDMS master to fabricate the PDMS microwells on the glass substrate. The PDMS negative master was then rendered hydrophilic by incubation with PLL-g-PEG for 30 minutes. The hydrophilic PDMS was used to prevent adhesion between the master and the freshly poured PDMS. The glass slides for the microwell fabrication were sonicated in isopropanol, rinsed in ultrapure water and dried under a stream of nitrogen. Final cleaning was performed in an oxygen plasma chamber to allow a good seal between the substrate and the PLL-g-PEG functionalized PDMS master.

### Various Possibilities for Microwell Fabrication Based on the Detection System

Based on the spectral signal (transmission or scattering intensity) to be measured the microwell fabrication (FIG. 6) can be optimized for a good signal-to-noise ratio. In case of the transmission mode the microwells 2 are optimum if narrow and tall (e.g. microwells that have the shape of long cylinders (cf. FIG. 6, left) with a diameter of e.g. between 100 nanometer to 1 millimeter, preferably between 1 micrometer and 100 micrometer, more preferably between 10 micrometer and 50 micrometer, and wherein the length or height is about one or several diameters long). This is advantageous, as there is more particle crowding along the optical path and this increases the signal recorded. FIG. 6 schematically shows the inner space or shape of typical microwells 2. For scattering spectrum measurements, where the detection focus is shallow compared to the transmission mode flat, wells 2 (high aspect ratio, cf. FIG. 6, right) are preferred (i.e. the height is only e.g. 1/10 to ⅔ of the diameter or less) facilitating the increase in particle crowding at the detection focus.

In order to test the performance of the proposed assay a model system was selected. The glass substrate 5 with microwell structure (cf., e.g., FIG. 7 or 5) was first plasma-cleaned to render the surface hydrophilic and then the wells 2 were filled with a mixture of Probe 1 and 2, wherein Probe 1 and Probe 2 are according to Table 3, functionalized gold nanoparticles 12, target 35 and 30% glycerol to prevent drying (cf. FIG. 8). These wells 2, i.e. their preferably upwardly facing openings 8, are covered with a coverslip 7 (cf. FIG. 8, top right). The complete spectral recording was done carefully without letting the wells 2 dry. The differences in spectra (cf. FIG. 8, bottom) in the presence (schematically shown in FIG. 8, right) and absence (schematically shown in FIG. 8, left) of target 35 were recorded and compared. The spectral measurements were conducted by a custom built microscope (Axiovert 200, Zeiss, Germany) with a spectrometer (SpectraPro 2150, PIXIS 400, Princeton Instruments, US). The data analysis and the control of the spectrometer were by a custom made program.

TABLE 3
DNA sequences of mRhoQ.

| Name | Modification | Sequence 5′-3′ |
|---|---|---|
| Probe 1 | Thiol-C6 (5′) | TTT TTC GTC AGT CAT GGG |
| (Seq. ID7) | | GTA |
| Probe 2 | Thiol-C6 (3′) | TTT ACC ACG GAG AAG CTT |
| (Seq. ID8) | | TTT |
| Target | None | TAC CCC ATG ACT GAC GTC |
| (Seq. ID9) | | TTC CTC ATA TGC TTC TCC |
| | | GTG GTA AA |

This sequence mRhoQ mentioned above is shown to be involved in neural regeneration in a previous work of Tanabe, K et al., The Small GTP-Binding Protein TC10 Promotes Nerve Elongation in Neuronal Cells, and Its Expression Is induced during Nerve Regeneration in Rats, The Journal of Neuroscience, 2000, 20(11): p. 4138-4144.

### Effect of Heating on Coupling of Nanoparticles:

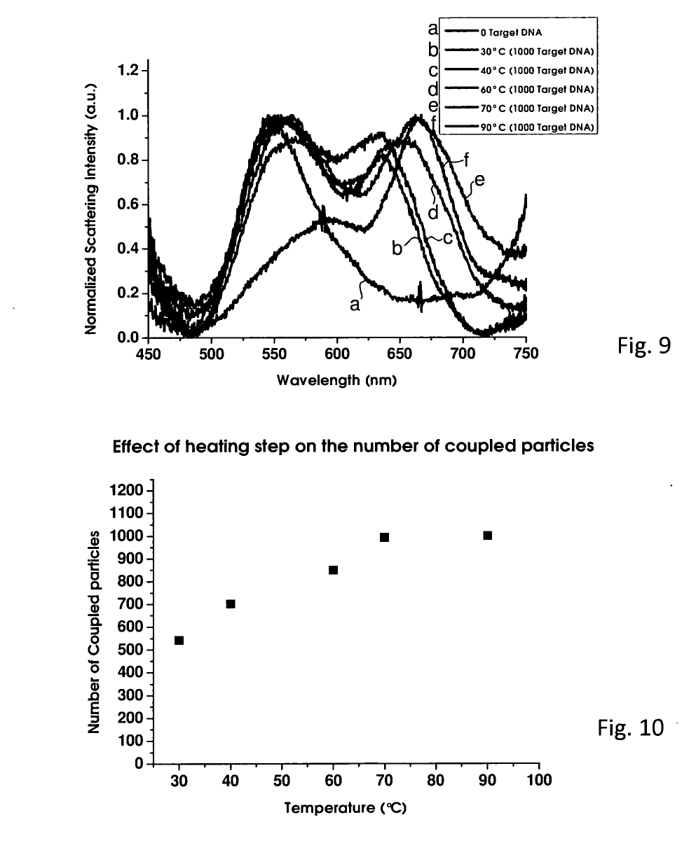

Experiments were done to determine the optimum temperature for the efficient hybridization of the target 35 to the gold probes 12. At room temperature there is a tendency for the DNA to form dimers among themselves, reducing the efficient hybridization between the probes 12 and the target 35. This can be overcome by heating the probes 12 together with the target 35 at elevated temperatures so there are more sites for hybridization. Around 1000 target DNA molecules 35 were added to 2000 gold colloids 12 producing particle pairs (1000 colloids of Probe 1 and 1000 colloids of Probe 2). The samples 30 were heated to different temperatures in the range from 30 to 90 degree Celsius, as specified in FIG. 9, and their scattering spectra were recorded at room temperature. It is observed that samples that were heated to 90° C. showed a higher secondary scattering peak, Peak 2 (P2), in the scattering spectra (cf. FIG. 9), which corresponds to an increased number of coupled particles 12. The number of coupled particles 12 was calculated from the area under the Peak 2 as follows:

$$Number of coupled particles = \frac{( 1000 * P 2 ( T ) )}{P 2 ( 90 ° C . )}$$

P2(T)—Area under Peak 2 at various Temperature;
P2(90° C.)—Area under Peak 2 heated to 90° C.

FIG. 10 shows the relation between the effect of the heating step or the temperature in degree Celsius and the number of coupled nanoparticles 12 used as detection probes 36.

### Dose Response Curve

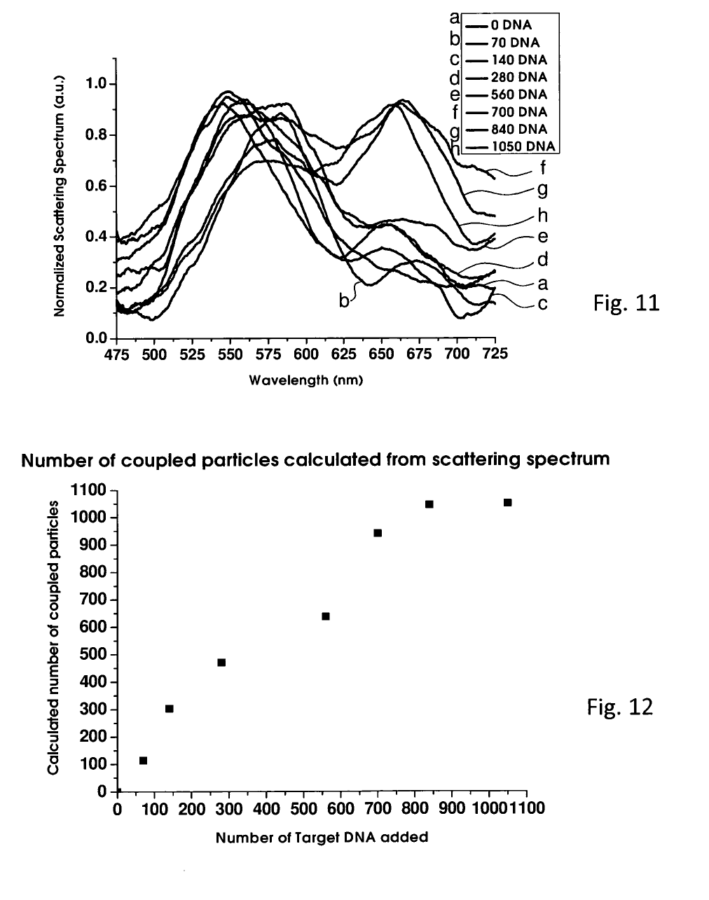

A similar experiment was performed to obtain the dose response curve. Various amounts of target DNA were added to 2000 nanoparticles and the scattering spectra (cf. FIG. 11) were recorded at room temperature after heating it to 90° C. FIG. 11 shows scattering spectra of gold nanoparticles 12 with varying numbers of target molecules 35, the respective amounts being up to 1050 DNA molecules as specified in the list in FIG. 11. The number of coupled particles 12 was calculated from the area under the Peak 2 as follows:

$$Number of coupled particles = \frac{( 1050 * P 2 ( C ) )}{P 2 ( 1050 DNA )}$$

P2(C)—Area under Peak 2 at various Concentrations;
P2(1050 DNA)—Area under Peak 2 for 1050 DNA.

FIG. 12 shows the relationship between the number of target DNA 35 added and the calculated number of coupled nanoparticles 12.

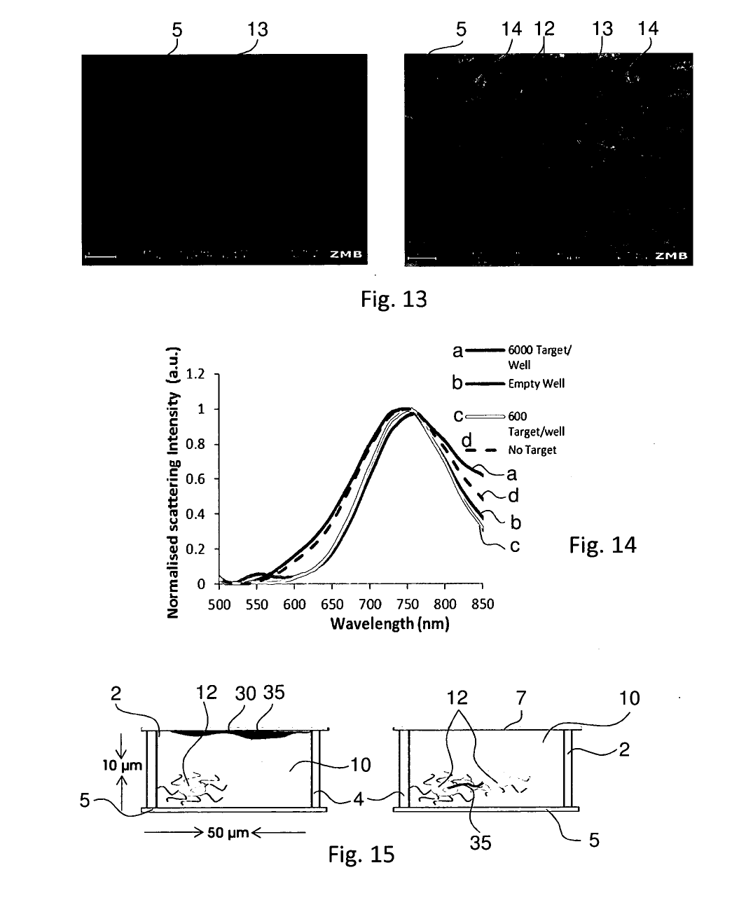

Another assay that can be made in such microwell system is the following: One probe (e.g. Probe 1) is fixed to the microwell substrate 5 and the other binds to the surface in the presence of the analyte. In this method the assay is confined to the surface of the microwell substrate 5 which has the advantage of bringing the signal generated by the assay 10 to the focal depth of the detection. This is also facilitates in neglecting the signal of the unbound probes 12 that will be out of the focal plane. Here, gold nanodisks 13 (about 110 nm diameter, about 30 nm thick, and about 300 nm apart) were used fabricated on an ITO surface by colloidal lithography (cf. FIG. 13). For this fabrication specific reference is made to: Hanarp, P., M. Kall, and D. S. Sutherland, Optical properties of short range ordered arrays of nanometer gold disks prepared by colloidal lithography. Journal of Physical Chemistry B, 2003. 107(24): p. 5768-5772), the disclosure of which is incorporated. FIG. 13 shows scanning electron microscope (SEM) images of gold nanodisks (13, 14) of 110 nm diameter on a substrate 5 with immobilized gold colloids 12 (50 nm diameter) due to DNA hybridization. The left image of FIG. 13 shows about 9×7 micrometer of the substrate 5. The right image of FIG. 13 is a SEM image with a width of about 2 micrometer and a 60° tilt angle. In the middle part of the right image in FIG. 13, the gold nanoparticles 12 are best visible. Situations are included in which, in general, said nanodisks 13 may have a larger or smaller diameter, e.g. in the range of 10 nanometer to 10 micrometer, more preferably 50 nanometer to 300 nanometer. The PDMS microwells 2 were fabricated on the nanodisk 13 substrate as mentioned above. The Probe 1 DNA with thiol group (Probe 1 will bind to the gold nanodisks 13, thereby producing functionalized nanodisks 14, target DNA, and the gold nanoparticles 12 functionalized with Probe 2 DNA were added to the microwell 2 and covered with coverslip 7. The spectral recordings (cf. FIG. 14) were performed as stated above, with focus to the surface of the nanodisks 14 to study the spectral shift due to change in the local refractive index around the nanodisks 14 (from buffer to gold colloid). FIG. 14 shows some exemplary spectral recording, i.e. the normalized scattering intensity in arbitrary units versus the light wavelength in nanometer, for a varying number of target ligands 35 in the microwell 2. The scattering peak in the red region (above 650 nm) is from the gold nanodisks and the peak in the blue region, i.e. at shorter wavelengths, of the spectrum is due to the binding of the gold nanoparticles to the nanodisks. The binding events also cause a slight shift in the near infra-red peak position.

This is due to the change in the local refractive index around the gold nanodisks.

A Microwell System to Detect mRNA Species in Single Cells

A common embodiment will be to perform similar assay on single or a small number of cells that are enclosed in a microwell 2 as shown in the schematics in FIG. 15. FIG. 15 schematically shows a microwell 2 which is provided on a substrate 5 and defined by walls 4. The microwell 2 is filled with a detection assay 10 including as detection probes 36 functionalized metal nanoparticles 12. Alternatively or additionally, other detection probes 36 may be used. Moreover, there is a flat substrate 6, i.e. a coverslip 7, on which the sample 30 with target ligand 35 is provided (left part of FIG. 15). The right part of FIG. 15 schematically shows the situation, after the detection probes 36, i.e. here the two metal nanoparticles 12, have coupled to the target ligand 35, wherein this coupling changes the optical response. The size of the microwell 2 is scaled down closer to the range of single cell (approx. 25 micrometer to 100 micrometer diameter). The size of the microwell 2, or the volume of an individual microwell 2, said volume is thus e.g. in the range of 10 picoliter to 1 nanoliter, preferably 100 picoliter to 500 picoliter, and more preferably about 200 picoliter, may be adapted to the size of the sample 30.

The present invention enables to detect and quantify the oligonucleotide of interest by using e.g. functionalized metal nanoparticles 12 as detection probes 36 to form a complex with the target ligand 35, which results in a considerable wavelength shift of the maximum in the localized surface plasmon resonance. Localized surface plasmon resonance of noble nanoparticles 11, 12 and their varied optical properties is a convenient and powerful means to enable quantification of analytes in a one pot assay. However, other proximity based mechanisms such as FRET (Fluorescence Resonance Energy Transfer) or Fluorescence Quenching could also be used for the same purpose. Fluorescent compounds may then be used as detection probes 36 instead of or additionally to the nanoparticles 11, 12. The present invention is indented to detect low copy numbers of DNA/RNA or very small quantities of DNA. A powerful application is to quantify the RNA from single cells in microwells, allowing systematic studies on live and fixed cells without the need for PCR or microarrays. The present invention enables direct quantification of these small amounts without the need to amplify them in PCR.

### LIST OF REFERENCE SIGNS

| | |
|---|---|
| 1 | Microwell array |
| 2 | Microwell |
| 3 | PDMS port |
| 4 | PDMS wall |
| 5 | Glass substrate |
| 6 | Flat substrate |
| 7 | Coverslip |
| 8 | Opening |
| 10 | Detection assay |
| 11 | Metal nanoparticle |
| 12 | Functionalized metal nanoparticle |
| 13 | Metal nanodisk |
| 14 | Functionalized nanodisk |
| 15 | Buffer |
| 30 | Sample with target ligand 35 |
| 31 | Reference assay |
| 35 | Target ligand in sample 30 |
| 36 | Detection probe |

1. A bioanalytical device consisting of comprising a microwell array configured to receive at least one assay component comprising a detection probe, and connected and/or or configured to be connected to a sample that is on a flat substrate to quantify an amount of a ligand in the sample using a detection mechanism, wherein the detection mechanism is based on a change in an optical property of the detection assay component upon contact with the ligand.

2. The bioanalytical device according to claim 1, wherein the detection probe is a metal nanoparticle based on or made of gold and/or or silver.

3. The bioanalytical device according to claim 1, wherein the microwell array comprises at least one microwell having a length, width and depth, or diameter and depth of 100 nanometer to 1 millimeter.

4. The bioanalytical device according to claim 1, wherein the detection probe is tagged with a thiolated DNA or with a thiolated oligonucleotide or nucleic acid, and wherein the ligand is a biomolecule.

5. The bioanalytical device according to claim 1, wherein the sample is a cell culture or a spotted microarray, and/or wherein the microwell array comprises a plurality of wells wherein at least two of the plurality of wells are filled with different assay components.

6. The bioanalytical device according to claim 1, wherein the assay component is selected based on the ligand and wherein the assay component comprises a compound which is suitable to induce lysis of the sample.

7. The bioanalytical device according to claim 1, wherein a first fraction of the detection probe is functionalized to couple to a first predefined part of the ligand by hybridization, and wherein a second fraction, of the detection probe is functionalized to couple to a second predefined part of the ligand by hybridization, wherein said second part of the ligand is located close to said first part of the ligand, such that the distance between the first fraction and the second fraction, which are coupled to the same ligand is small, so that the coupled first fraction and coupled second fraction are coupled optically.

8. The bioanalytical device according to claim 3, wherein the microwell comprises a bottom that is decorated with a nanodisk.

9. A method for detecting molecules using the bioanalytical device according to claim 3, comprising determining an optical property of at least a part of the assay component and the sample.

10. The method according to claim 9, wherein said flat substrate is a coverslip being used for covering the microwell, wherein the sample is provided on said coverslip, and wherein the sample is in communication with said assay component upon covering the microwell with the coverslip.

11. The method according to claim 9, wherein measuring the optical spectra includes measuring a surface plasmon resonance of the detection.

12. The method according to claim 9, wherein the optical property includes a transmission or a scattering intensity of light transmitted through or scattered by the assay component and sample.

13. The method according to claim 9, wherein the optical property is a fluorescent signal further comprising measuring a change in said fluorescent signal changes upon coupling between the target ligand and the detection probe.

14. The method according to claim 10, wherein the microwell array comprises a plurality of microwells, and wherein each microwell can be measured individually and/or wherein the detection mechanism is imaging based, therefore making parallel detection of multiple samples possible.

15. The bioanalytical device according to claim 1, wherein the detection probe comprises metal nanoparticle based on or made of gold and/or silver, wherein the nanoparticle has a diameter of up to and inclusive of 50 nanometer, or wherein the detection probe comprises a fluorescent compound based on or made of a dye and/or a protein.

16. The bioanalytical device according to claim 3, wherein the length, width and depth or diameter and depth of the microwell is between 10 micrometer and 50 micrometer, wherein said microwell has a cylindrical shape.

17. The bioanalytical device according to claim 4, wherein the detection probe can couple to a predefined part of said ligand by hybridization, and wherein the ligand is a protein, RNA, DNA, an oligonucleotide, a carbohydrate, or a lipid, a small molecule, or a cell fragment.

18. The bioanalytical device according to claim 7, wherein the first fraction represents 40% up to 60% of the detection probe and comprises a predefined thiolated oligonucleotide, wherein the second fraction represents 60% down to 40%, of the detection probe and comprises a predefined thiolated oligonucleotide, and wherein said distance being smaller than or equal to 5 nanometer.

19. The bioanalytical device according to claim 8, wherein said nanodisk is based on or made of gold and/or silver, wherein said nanodisk has a diameter of 50 nanometer to 300 nanometer, wherein said nanodisk has a distance of about 100 to 500 nanometer from a second nanodisk, wherein said nanodisk is functionalized with a thiolated oligonucleotide, such that the functionalized nanodisk is adapted to couple to a first predefined part of the ligand, by hybridization, wherein at least part of the detection probe is functionalized with a predefined thiolated oligonucleotide, such that said functionalized detection probe is coupled to and adapted to a second predefined part of the ligand, by hybridization, wherein said second part of the ligand is located close to said first part of the ligand, such that a distance between said nanodisk and said detection probe coupled to the same ligand is smaller than or equal to 5 nanometer.

20. A method for detecting molecules using the bioanalytical device according to claim 9, wherein said optical property is a color that is visible by the naked eye or said optical property being an optical spectra, wherein a spectrometer means are applied for measuring of said optical spectra, wherein said color or said optical spectra are determined, at room temperature, after the ligand to be quantified in the sample has coupled to the detection probe, after heating the assay component and the sample, further comprising determining a quantity of the ligand in the sample is determined by comparing said color or said optical spectra with a reference, wherein said reference is a color of or an optical spectra recorded for a reference assay with a known ligand quantity.

21. The method according to claim 10, wherein said sample is a cell culture, a spotted microarray, a single cell, or a small number of cells; wherein said sample is provided in a dried state; and wherein the sample couples with the detection probe upon covering the microwell with the coverslip; and wherein the target ligand in the sample is a protein, RNA, DNA, an oligonucleotide, a carbohydrate, or a lipid, a small molecule, or a cell fragment.

22. The method according to claim 11, wherein the optical property is measured in a light wavelength range from 400 nanometer to 800 nanometer; further comprising determining a maximum of said surface plasmon resonance, and comparing with similar optical spectra from the reference assay includes comparing said maximum, wherein a wavelength shift of said maximum is used as a measure for the quantity of the ligand in the sample.

23. The method according to claim 12, wherein the microwell is optimized to have a shape of a long cylinder for transmission intensity measurements or to have a disk-like shape for scattering intensity measurements.

24. The method according to claim 14, wherein each microwell is measured individually, at the same time.
